(19) United States
(12) Patent Application Publication
(10) Pub. No.: US 2019/0304023 A1
(43) Pub. Date: Oct. 3, 2019

(54) HEALTHCARE BENEFITS PLAN RECOMMENDATION

(71) Applicant: DZee Solutions, Inc., Centennial, CO (US)

(72) Inventors: Ashwin K. Pingali, Parker, CO (US); Bipin Agarwal, Englewood, CO (US)

(21) Appl. No.: 16/118,142

(22) Filed: Aug. 30, 2018

Related U.S. Application Data
(60) Provisional application No. 62/651,632, filed on Apr. 2, 2018.

(51) Int. Cl. G06Q 40/08; G16H 10/60; G06Q 10/10

(52) U.S. Cl. CPC G06Q 40/08; G16H 50/20; G06Q 10/105; G16H 10/60

(57) ABSTRACT

The disclosed technology provides a method for generating quantitative recommendations for healthcare benefits plans using hierarchical layered graphs. One or more nodes are identified in the hierarchical layered graphs. The hierarchical layered graphs store historical healthcare claims data. The one or more nodes are identified based on contextual data associated with an employee and on similarities between the contextual data associated with the employee and node contextual data associated with the node. One or more paths are identified among the one or more identified nodes. A plurality of healthcare plans are scored using the hierarchical layered graphs by applying contextual data associated with the plurality of healthcare plans to the identified paths. A subset of the plurality of scored healthcare plans are identified and recommended to the employer or the employee.

### CROSS-REFERENCE TO RELATED APPLICATIONS

The present application claims priority to pending U.S. Provisional Patent Application Ser. No. 62/651,632 entitled “Healthcare Plan Recommendation,” filed on Apr. 2, 2018, which is specifically incorporated by reference herein for all that it discloses and teaches.

### BACKGROUND

Healthcare cost forecasting mechanisms for individuals, family units, and groups of family units (e.g., employers having multiple family units covered in a benefits plan) can assist financial planning for these healthcare costs. Calculators provide models and indicators for healthcare expenses based on determinate factors, such as Medicare premiums. However, such models and indicators are static in nature, and do not take into account contextual information about individuals, families, or employers. These limitations can result in relatively inaccurate projections when used to calculate costs over long periods of time. Further, it is time consuming and difficult to use such models to select a healthcare benefits plan from large number of available healthcare benefits plans, especially when taking into account individualized requirements for individuals, families, and employers. Inaccuracies in financial projections can make such projections unreliable and can make it difficult for individuals and employers to efficiently plan for healthcare costs. Similar issues may arise when selecting any type of benefits plan, such as life insurance, homeowner's insurance, etc.

### SUMMARY

The described technology addresses one or more of the foregoing problems by providing a method for generating quantitative recommendations for healthcare benefits plans using hierarchical layered graphs. One or more nodes are identified in the hierarchical layered graphs. The hierarchical layered graphs store historical healthcare claims data. The one or more nodes are identified based on contextual data associated with an employee and on similarities between the contextual data associated with the employee and node contextual data associated with the node. One or more paths are identified among the one or more identified nodes. A plurality of healthcare plans are scored using the hierarchical layered graphs by applying contextual data associated with the plurality of healthcare plans to the identified paths. A subset of the plurality of scored healthcare plans are identified and recommended to the employer or the employee.

### BRIEF DESCRIPTION OF DRAWINGS

FIG. 1 illustrates a block diagram of an example healthcare benefits plan recommendation engine.

FIG. 2 illustrates an example hierarchical layered graph for recommending a healthcare benefits plan.

FIG. 3 illustrates example operations for generating healthcare recommendations using a recommendation engine.

FIG. 4 illustrates example operations for generating healthcare recommendations using a recommendation engine.

FIG. 5 illustrates a block diagram of an example computer system suitable for implementing one or more embodiments of the disclosed technology.

### DETAILED DESCRIPTION

Implementations illustrated and described herein utilize a rich dataset of healthcare interaction data (e.g., doctor visits, transactions, claims) implemented as a heterogeneous information network (e.g., a hierarchical layered graphs) to generate healthcare insurance plan recommendations for employers and/or employees. The implementations combine reinforcement algorithms, path trajectories, graph layering, and decision-making techniques to provide customized recommendations based on information about the employer and/or employee.

Similar methods may be used to generate recommendations for other types of benefits plans in addition to healthcare benefits plans. For example, information about homeowners and homes may be used to help homeowners select a home insurance policy. Similarly, health data and life expectancy data may be used to help consumers select life insurance benefits policies.

Information about the employer and one or more employees is collected. In some implementations, employee intents and employer intents are obtained directly from the employee or employer. In other implementations, the information collected about the employer and employees is used to infer one or more intents. An employer intent may be any objective that the employer is looking to achieve through the selection of healthcare plans. For example, for a risk conscious employer, the recommendation engine may infer that the employer has an intent of low cost. For an employer that is trying to recruit elite employee talent, the recommendation engine may infer that the employer has an intent of offering good benefits. Similarly, an employee intent may be any objective that an employee is looking to achieve through the selection of a healthcare plan. A single employee with no dependents may have different intents (e.g., low cost) than a married employee with one or more dependents (e.g., best benefits). The intents may be stored as one or more nodes in a hierarchical layered graph.

Healthcare plans are associated with a plurality of contextual data. Contextual data may include the covered benefits, plan offering, expenses (e.g., out of pocket, deductibles), provider networks, employer/employee risks, claim processing, special needs, etc. Information about the employees and the intents are utilized to identify similar nodes (e.g., people and people interaction) in the heterogeneous information network of healthcare interaction data. The identification may include clustering, ranking, finding similarities, etc. For example, a thirty-year-old male is clustered near other males around the age of thirty years old. When a subset of data points (e.g. nodes) are identified that are similar to the subject employee, the recommendation engine runs a plurality (e.g., millions) of simulations to identify likely trajectories for the individual.

Example trajectories include a sequence of paths through the graph indicating doctor visits, prescription purchases, etc. over an extended period of time. The simulations account for the contextual data associated with the plurality of healthcare plans. Thus, the recommendation engine determines possible costs associated with different plans, possible coverage, provider networks, risks, etc. Depending on the amount of information known by the employee, the simulations may be run in a personalized specific context (e.g., with detailed data about the employee), a localized context (e.g., with some amount of data about the employee), or in a globalized context (e.g., with demographic data about the employee). Similar simulations are run with the group of employees for the employer. Thus, specific costs and benefits outcomes may be generated based on the trajectory of the group and the contexts associated with the healthcare plans.

In one implementation, the recommendation engine outputs a subset of ranked healthcare plans based on the simulated trajectories, contexts associated with the plans, the intents of the employer and/or the employee, etc. Furthermore, the recommendation engine outputs specifically identified plans for each of the employees based on the simulated trajectories, contexts associated with the plans, the intents of the employee, etc. User (e.g., employer/employee) interactions associated with recommended plans may be collected and utilized to provide more information of how the recommendations were processed and/or to enrich the dataset associated with intents. Furthermore, as employer/employees interact with selected plans over time, the hierarchical layered graphs including the intent nodes are updated via reinforcement learning methodology. Thus, the system tracks whether the intents are satisfied (e.g., cost remains low) over time.

In one implementation, the hierarchical layered graphs include temporal nodes identifying points in times when specific healthcare interaction data occurs, which are used to specify temporal dependencies. Thus, relative interactions are utilized to simulate trajectories for specific individuals and groups of individuals based on information about the individuals. Furthermore, the graph is self-reinforcing in that it is able to intelligently identify missing data and fill in the data. For example, if a specific interaction has a deductible of $0, then the graph infers that previous interactions had a non-zero value and fills in such data. Furthermore, because the graph utilizes layering techniques, the graph provides index free search and fast traversal for trajectories. A number of path trajectories are identified with probabilities associated with those trajectories using predictive intelligence techniques and machine learning models.

The hierarchical layered graphs provide an improved method for storing historical healthcare interaction data for the purposes of recommending healthcare benefits plans. The highly complex hierarchical layered graphs allow for the storage of vast amounts of data in a format that is easily accessible for machine learning applications. Further, a computer may traverse paths within the hierarchical layered graphs quickly, providing the ability to traverse a large number (e.g., millions) of paths in seconds to provide a quick healthcare benefits plan recommendation.

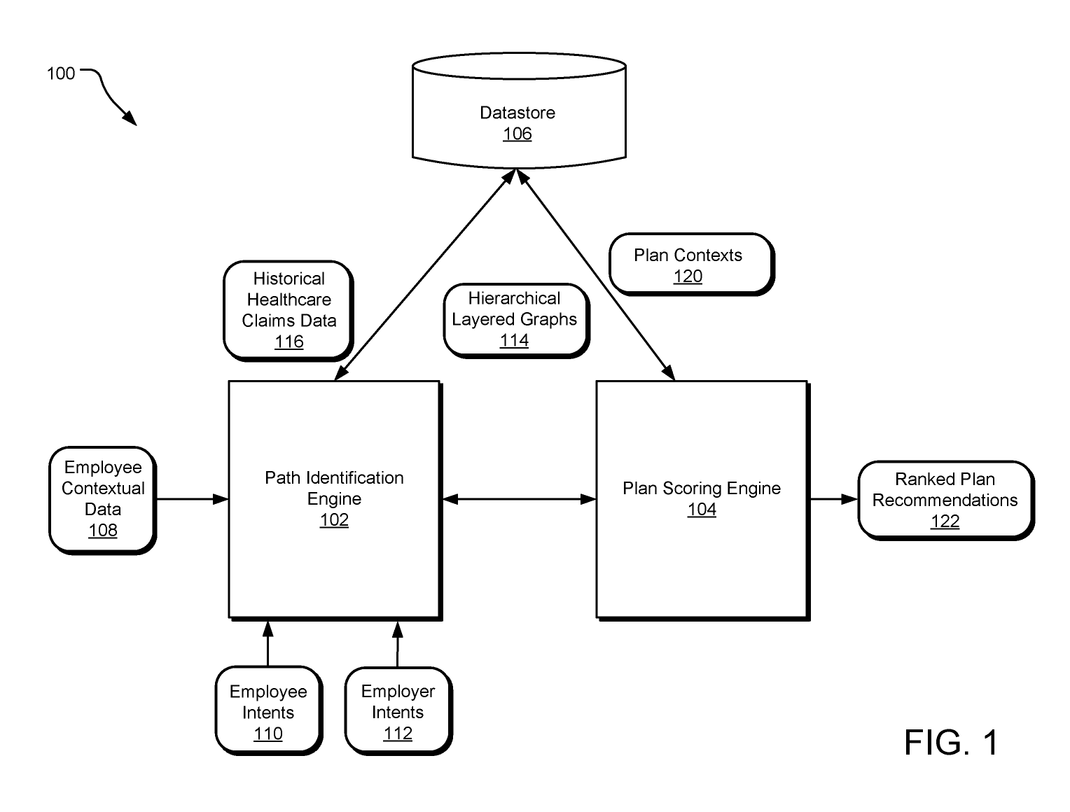

FIG. 1 illustrates a block diagram of an example system 100 for generating healthcare benefits plan recommendations. The system 100 includes a path identification engine 102 and a plan scoring engine 104. The path identification engine 102 and the plan scoring engine 104 may both communicate with a datastore 106. The datastore 106 may be located locally, on a computing device including the path identification engine 102 and the plan scoring engine 104, or remotely, such as on a cloud computing device. When the datastore 106 is located remotely, the path identification engine 102 and the plan scoring engine 104 may be communicatively connected to the datastore 106 through, for example, a WiFi or cellular data connection.

The path identification engine 102 receives inputs of employee contextual data 108, employee intents 110, and employer intents 112. Employee contextual data 108 may be any information about an employee or group of employees that may be relevant to selecting a health plan. For example, employee contextual data 108 may include the age and sex of employees, whether employees have dependents who will also be on the selected health care plan, or the employee's ZIP code. In some implementations, employee contextual data 108 may also include detailed information such as health status, medical history, lifestyle, and past healthcare claims data. Employee intents 110 and employer intents 112 may include information about what objectives the employer and employee have regarding health care plans. For example, employer intents 112 may indicate that the employer wants to find the health care plan that is the lowest cost to the employer. Or, an employer whose objective is to provide the best benefits for employees may have an employer contextual data indicating that the employer wants to find the healthcare plan that is the lowest cost to employees. Similarly, employee intents 110 may indicate that the employee is looking for the healthcare plan with the lowest out of pocket cost or the broadest coverage.

In some implementations, the path identification engine 102 may receive employee intents 110 and employer intents 112 directly from an employer or employee. For example, the path identification engine 102 may generate a series of questions for an employer or employee to answer and may generate employee intents 110 and employer intents 112 based on the received answers. In other implementations, the path identification engine includes an intent module (not shown). The intent module receives information about an employer and the employee to determine the employer intents 112 and the employee intents 110. The intent manager may utilize an intent graph implemented as a hierarchical layered graph to identify the employer intents 112 or the employee intents 110.

The path identification engine 102 also receives hierarchical layered graphs 114 built from historical healthcare claims data. In the implementation shown in FIG. 1, the hierarchical layered graphs 114 are stored on the datastore 106 and communicated directly to the path identification engine 102. In other implementations, the hierarchical layered graphs 114 may be built using a network building engine (not shown) and may be communicated from the network building engine to the path identification engine 102. The building phase of the hierarchical layered graphs 114 represents the training of the path identification engine 102. As the hierarchical layered graphs 114 are built, meaning more data is added to the hierarchical layered graphs 114, the path identification engine 102 becomes more accurate at predicting healthcare system interactions for individuals or groups of individuals.

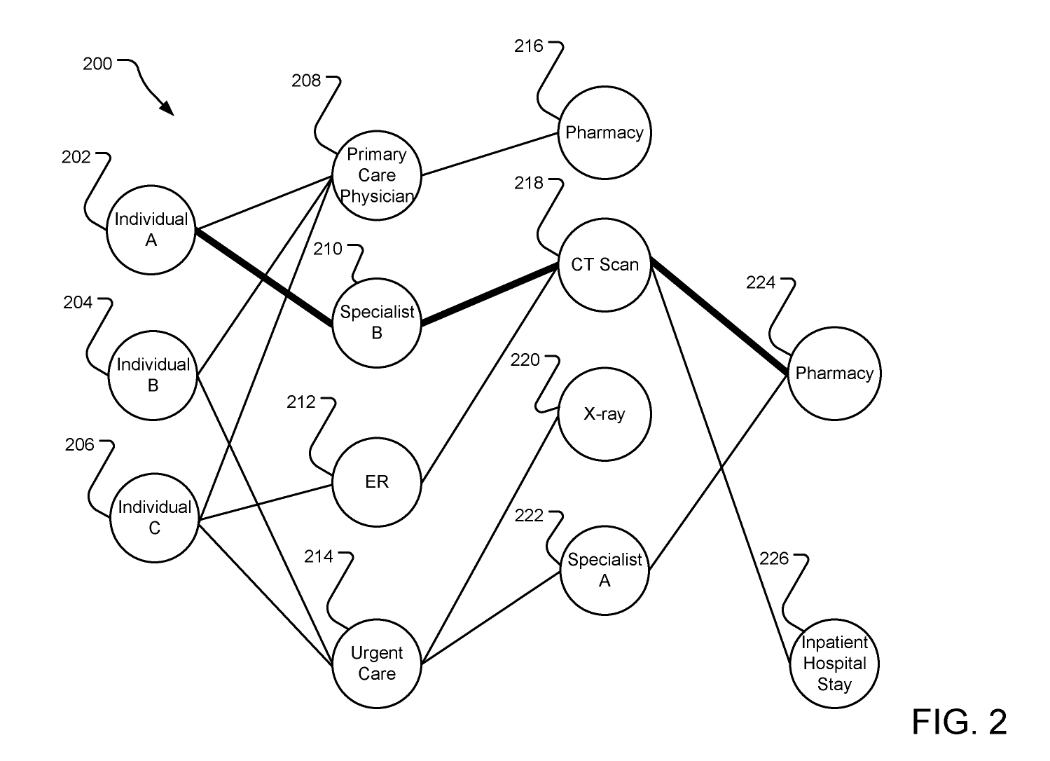

The hierarchical layered graphs 114 consist of a plurality of nodes. The nodes in the hierarchical layered graphs 114 may represent, for example, a covered individual, a particular healthcare facility, a particular physician, or a particular pharmacy. Nodes in the hierarchical layered graphs 114 are connected by edges when there is an interaction between the nodes. For example, if a covered individual receives care from a physician, the nodes representing the covered individual and the physician will be connected in the heterogeneous information network 114. The hierarchical layered graphs 114 are built and trained using historical healthcare claims data 116. As more historical healthcare claims data 116 is added to the hierarchical layered graphs 114, more nodes are created, and more edges are added between nodes to represent healthcare claims and interactions with healthcare systems. As the hierarchical layered graphs 114 are built, clustering may be used to simplify the hierarchical layered graphs 114. For example, nodes that are similar may be clustered together. Nodes that are clustered may represent, for example, similar individuals, similar healthcare providers, or similar healthcare facilities. The hierarchical layered graphs 114 are discussed with more detail with reference to FIG. 2.

Once the hierarchical layered graphs 114 are built, the path identification engine 102 identifies one or more nodes in the hierarchical layered graphs 114 based on information associated with an employer and one or more employees. The identified nodes are identified based on similarities between the nodes and the employee information 108. For example, a given employer may provide employee information 108 for eight employees. Eight nodes may be identified in the hierarchical layered graphs 114, where each of the eight nodes represents a covered individual that is similar to the employee. For example, a covered individual may be the same age and sex as the employee and may reside in the same ZIP code.

The path identification engine 102 then identifies one or more paths between the identified nodes in the hierarchical layered graphs 114 based on the employee intents 110 and the employer intents 112. In some implementations, the path identification engine 102 may identify the most likely path in the hierarchical layered graphs 114 for each employee. In other implementations, employee intents 110 or employer intents 112 may indicate that the path identification engine 102 identifies the least likely path in the hierarchical layered graphs 114. For example, when the employer intents 112 indicate that the employer wishes to provide extremely robust healthcare coverage to its employees, the path identification engine 102 may identify a path that results in the most interactions with the healthcare system for a similar individual.

Once the path identification engine 102 has identified paths between the identified nodes in the hierarchical layered graphs 114, the path identification engine 102 communicates the hierarchical layered graphs 114 including the identified paths to the plan scoring engine 104. The plan scoring engine 104 generates ranked plan recommendations based on plan contexts 120 and the hierarchical layered graphs 114 including the identified paths. The plan contexts 120 include data about the costs to the employer and employee for a multitude of healthcare plans for every healthcare interaction represented in the hierarchical layered graphs 114. The plan scoring engine 104 uses the plan contexts 120 to calculate the cost of the identified paths on the hierarchical layered graphs 114 for a variety of healthcare plans. The plan scoring engine 104 then ranks the healthcare plans based on cost. In some implementations, the healthcare plans may be ranked solely on the cost to the employer, with less costly plans ranked higher than more costly plans. In other implementations, the healthcare plans may be ranked based on the employee intents 110 and the employer intents 112. For example, when the employee intents 110 indicates that the employee wants to find the plan with the lowest deductible, the healthcare plans may be ranked solely on deductibles.

In some implementations, two plan rankings may be produced, where one ranking ranks healthcare plans for the employer based solely on the employer intents 112. Another ranking of plans may be generated for the employee based on the employee intents 110. In some implementations, the employee ranking may be generated only for plans that have been previously selected by the employee's employer.

FIG. 2 illustrates an example hierarchical layered graph 200. The hierarchical layered graph 200 is made up of a plurality of nodes and edges connecting the nodes. In the hierarchical layered graph 200, nodes 202, 204, and 206 each represent an individual. Remaining nodes 208, 210, 212, 214, 216, 218, 220, 222, 224, and 226 represent entities in the healthcare system, such as individual providers, healthcare facilities, and treatments. In other implementations, nodes may include temporal information, specific diagnoses, or other information relevant to healthcare claims.

Nodes in the hierarchical layered graph 200 are connected by edges, indicating that an interaction has occurred between two nodes. For example, node 202 represents Individual A. Individual A's interactions with the healthcare system are represented by an edge connecting the node 202 with the node 208 and an edge connecting the node 208 with the node 210. These edges indicate that Individual A saw the primary care physician represented by the node 210 and the specialist represented by the node 218. Similarly, edges connected the node 204 to other nodes in the hierarchical layered graph 200 represent Individual B's interactions with the healthcare system.

Other edges represent possible paths, or trajectories, through the healthcare system for individuals. For example, the bolded edges represent a possible trajectory for Individual A. Edges can also include information about the frequency of a particular interaction or trajectory. For example, the edge connecting the node 208 with node 216 indicates that, for all of the individuals currently included in the hierarchical layered graph 200, every individual who saw the primary care physician represented by the node 208 then used the pharmacy represented by the node 216. If, for example, an individual went to the primary care physician represented by the node 208 and then had no further action, a node may be included in the hierarchical layered graph 200 to indicate the lack of further interaction with the healthcare system.

When the hierarchical layered graph 200 is built, edges may be given a different weight depending on how many times the edge is traversed when building the hierarchical layered graph 200. For example, because all of the individuals included in the hierarchical layered graph 200 visited the primary care physician represented by the node 208 and then proceeded to use the pharmacy represented by the node 216, the edge between the node 208 and the node 216 would be given a higher weight. Edges that are traversed less frequently would be given a lower weight.

In addition to the hierarchical layered graph 200, sub-graphs may exist, displaying the properties of nodes in the hierarchical layered graph 200. For example, a sub-graph may exist that divides the individuals represented by the nodes 202, 204, and 206, along with other individuals, into different health grades. Additionally, the data displayed in the hierarchical layered graph 200 may be combined with other data or displayed differently in related hierarchical layered graphs. For example, additional hierarchical layered graphs related to the hierarchical layered graph 200 may include nodes for companies or families of covered individuals. Additional graphs may also include graphs representing specific health grades or centered around particular healthcare providers.

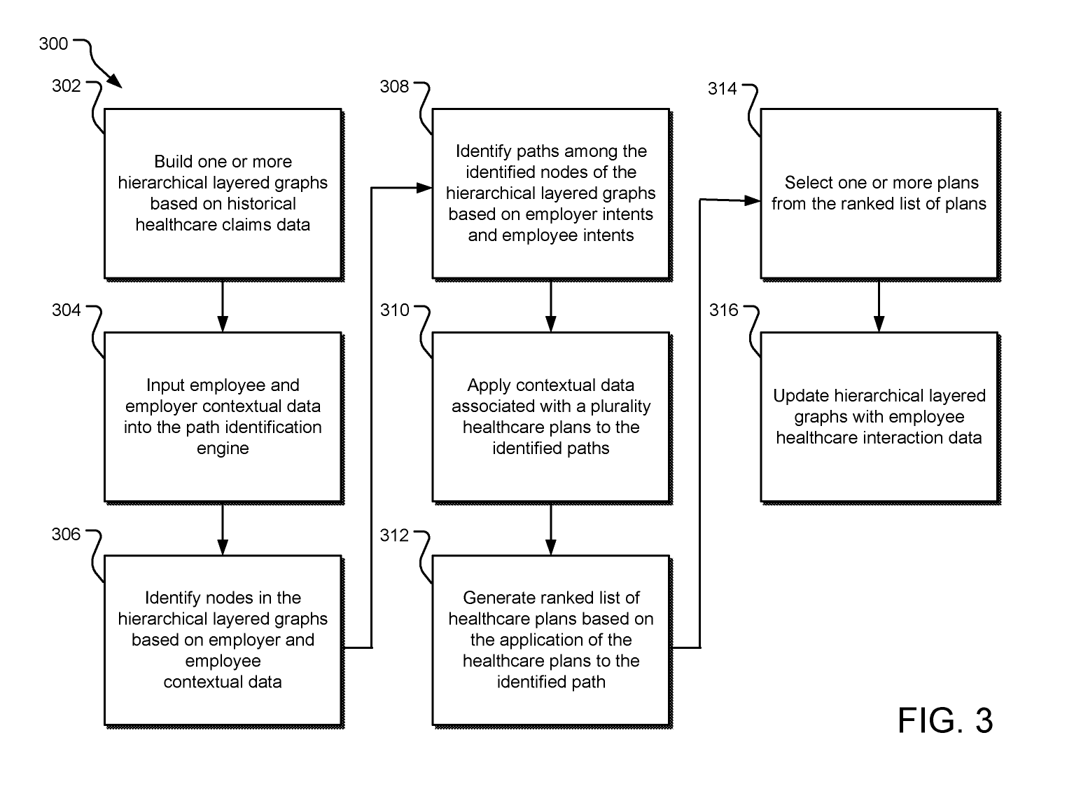

FIG. 3 illustrates example operations 300 for generating healthcare recommendations using a recommendation engine. A building operation 302 builds one or more hierarchical layered graphs based on historical healthcare claims data. The building operation 302 represents the training stage of the hierarchical layered graphs. The hierarchical layered graphs include nodes connected by edges, representing interactions within a healthcare system. For example, a hierarchical layered graph may include nodes representing individuals connected by edges to nodes representing healthcare facilities or healthcare providers. Multiple hierarchical layered graphs may be created in the building operation 302 representing the same or different information. For example, additional hierarchical layered graphs may display similar information, but may include nodes for families of individuals or for an employer. Additional sub-graphs may be generated during the building operation 302.

An inputting operation 304 inputs employee contextual data and employer contextual data into the path identification engine. The employer contextual data and employee contextual data may be input directly through a user interface. The employer contextual data may include information about the employer, including the number of employees, coverage needs, and other employer data. The employee contextual data may include basic demographic information about employees and other individuals, such as the employee's family members, that may be covered under the employee's healthcare plan. The employee contextual data may also include other information about individuals that may be covered under the employee's healthcare plan, such as health status information, previous medical claims, and lifestyle information.

A first identifying operation 306 identifies nodes in the hierarchical layered graphs based on employer and employee contextual data. The nodes may be identified based on similarities between either the employer and the nodes or the employee and the nodes. For example, in a hierarchical layered graph with nodes representing employers, the nodes identified in the first identifying operation 306 may represent an employer with a similar number of employees who fit into similar age and sex demographics. The nodes may be identified based on node contextual data associated with the nodes. Node contextual data may include any additional information about the person, entity, or treatment represented by the node. For example, a node representing an individual may be associated with node contextual data including demographic and health information about the individual represented by the node. Additionally, identified nodes include nodes relevant to a node representing a similar employer, such as, for example, specific providers, types of providers, medications, or healthcare facilities. Similarly, in a hierarchical layered graph with nodes representing covered individuals, the nodes identified in the first identifying operation 306 may represent covered individuals with similar characteristics to the employees of the employer, based on employee information. Nodes relevant to the similar covered individuals are also identified in the first identifying operation 306. Further, the first identifying operation 306 may identify multiple nodes in multiple hierarchical layered graphs.

In some implementations, the employee contextual data may include only employee demographic information, such as an employee's age, sex, ZIP code, and marital status. Accordingly, the nodes identified in the first identifying operation 306 will be nodes associated with individuals with similar demographic information. In other implementations, the employee contextual data may include employee health information, such as health status of an employee, information about the employee's lifestyle, and historical claims data for the employee. Accordingly, the nodes identified in the first identifying operation 306 will include nodes associated with individuals with similar demographic and health information. The inclusion of employee health information may make predictions more tailored to the employee's situation and health concerns.

A second identifying operation 308 identifies paths among the identified nodes of the hierarchical layered graphs based on contextual data. The second identifying operation 308 may identify paths between the identified nodes and other nodes in the hierarchical layered graphs based on employee intents or employer intents, or a combination of employee intents and employer intents. In some implementations, the paths may represent the most likely interactions with the healthcare system for a given individual or group of individuals. For example, the second identifying operation 308 may identify paths with higher weights. In other implementations, the identified paths may represent outlier situations. For example, the second identifying operation 308 may identify the paths with lower weights or paths that result in the most interaction with the healthcare system.

An applying operation 310 applies contextual data associated with a plurality of healthcare plans to the identified paths. The contextual data associated with the plurality of healthcare plans includes data about the costs to the employer for interactions with the healthcare system under each healthcare plan. The contextual data associated with the plurality of healthcare plans may also include information about the costs to the employee for interactions with the healthcare system under each healthcare plan. The applying operation 310 applies the contextual data to the paths representing interactions with the healthcare system to find the costs of the identified paths using each of the plurality of healthcare plans.

A generating operation 312 generates a ranked list of healthcare plans based on the application of the healthcare plans to the identified paths. The ranked list of healthcare plans may also be generated based on employer contextual data or employee contextual data, or on a combination of employer contextual data and employee contextual data. A selecting operation 314 selects one or more plans from the ranked list of plans.

An updating operation 316 updates the hierarchical layered graphs with employee healthcare interaction data. The updating operation 316 updates the hierarchical layered graphs by creating new nodes for the employees covered by a specific healthcare plan after the employer or employees have selected the plans from the ranked list of plans in the selecting operation 314. New edges are created representing the employee's actual interactions with the healthcare system. Weights of existing edges are updated based on the employee's actual interactions with the healthcare system. The hierarchical layered graphs become more robust and accurate predictors of healthcare system interactions as a result of the updating operation 316.

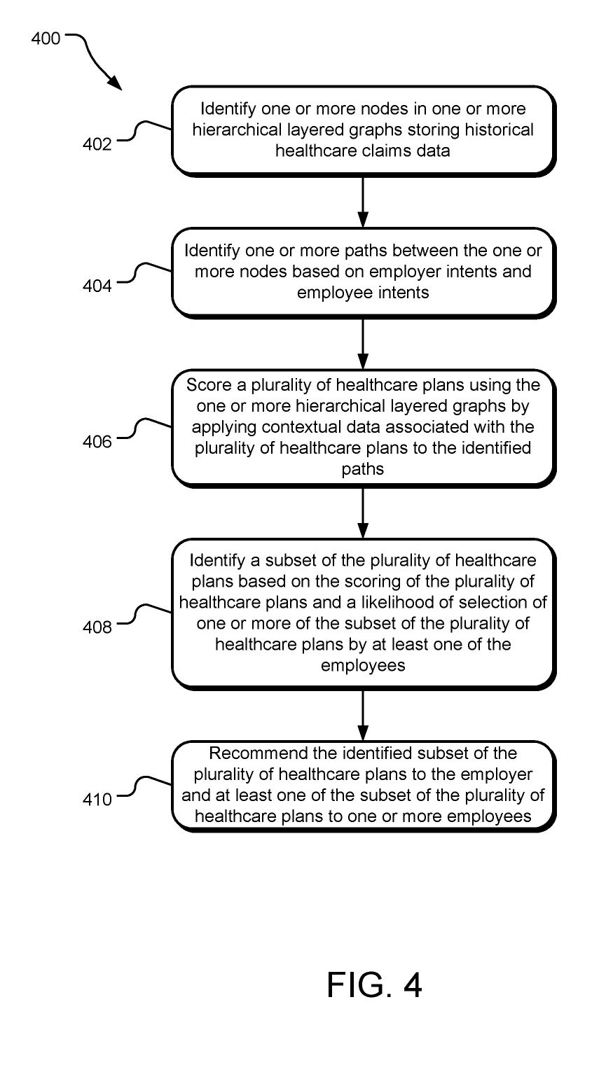

FIG. 4 illustrates example operations 400 for generating healthcare recommendations using recommendation engine. A first identifying operation 402 identifies one or more nodes in one or more hierarchical layered graphs storing historical healthcare claims data. The identified nodes are identified based on contextual data associated with the employer and employees associated with the employer. The nodes may be identified based on similarities between either the employer and the nodes or the employee and the nodes. For example, in a hierarchical layered graph with nodes representing employers, the nodes identified in the first identifying operation 402 may represent an employer with a similar number of employees who fit into similar age and sex demographics. Additionally, identified nodes include nodes relevant to a node representing a similar employer, such as, for example, specific providers, types of providers, medications, or healthcare facilities. Similarly, in a hierarchical layered graph with nodes representing covered individuals, the nodes identified in the first identifying operation 306 may represent covered individuals with similar characteristics to the employees of the employer, based on employee information. Nodes relevant to the similar covered individuals are also identified in the first identifying operation 402. Further, the first identifying operation 306 may identify multiple nodes in multiple hierarchical layered graphs.

In some implementations, the employee contextual data may include only employee demographic information, such as an employee's age, sex, ZIP code, and marital status. Accordingly, the nodes identified in the first identifying operation 402 will be nodes associated with individuals with similar demographic information. In other implementations, the employee contextual data may include employee health information, such as health status of an employee, information about the employee's lifestyle, and historical claims data for the employee. Accordingly, the nodes identified in the first identifying operation 402 will include nodes associated with individuals with similar demographic and health information. The inclusion of employee health information may make predictions more tailored to the employee's situation and health concerns.

A second identifying operation 404 identifies one or more paths among the one or more nodes based on employer intents or employee intents of the one or more employees. The employer and employee intents may be used to determine whether the identified paths should traverse the most likely path among the identified nodes or an outlier path among the identified nodes. For example, when an employer intent is to provide the lowest cost coverage to its employees, the second identifying operation 404 may identify the most likely path among the identified nodes. Alternatively, when an employer intent is to provide robust coverage, the second identifying operation 404 may identify an outlier path between the identified nodes that includes a large amount of interaction with the healthcare system.

A scoring operation 406 scores a plurality of healthcare plans using the one or more hierarchical layered graphs by applying contextual data associated with the plurality of healthcare plans to the identified paths. The contextual data associated with the plurality of healthcare plans includes data about the costs to the employer for interactions with the healthcare system under each healthcare plan. The contextual data associated with the plurality of healthcare plans may also include information about the costs to the employee for interactions with the healthcare system under each healthcare plan. The scoring operation 406 applies the contextual data to the paths representing interactions with the healthcare system to find the costs of the identified paths using each of the plurality of healthcare plans.

A third identifying operation 408 identifies a subset of the plurality of healthcare plans based on the scoring of the plurality of healthcare plans and a likelihood of selection of one or more of the subset of the plurality of healthcare plans by at least one of the employees. The plurality of healthcare plans may be identified based on employer and employee intents. For example, when employee intents for all of the employees of a certain employer indicate the desire for low out of pocket costs, a healthcare plan with high out of pocket costs would likely not be identified in the third identifying operation 408.

A recommending operation 410 recommends the identified subset of the plurality of healthcare plans to the employer and at least one of the identified subset of the plurality of healthcare plans to one or more employees. In some implementations, the recommended plans may be recommended based on predicted cost of the recommended plans. Predicted cost may include, for example, predicted cost for the employer, predicted out of pocket costs for the employee, or actual premium costs for the employee.

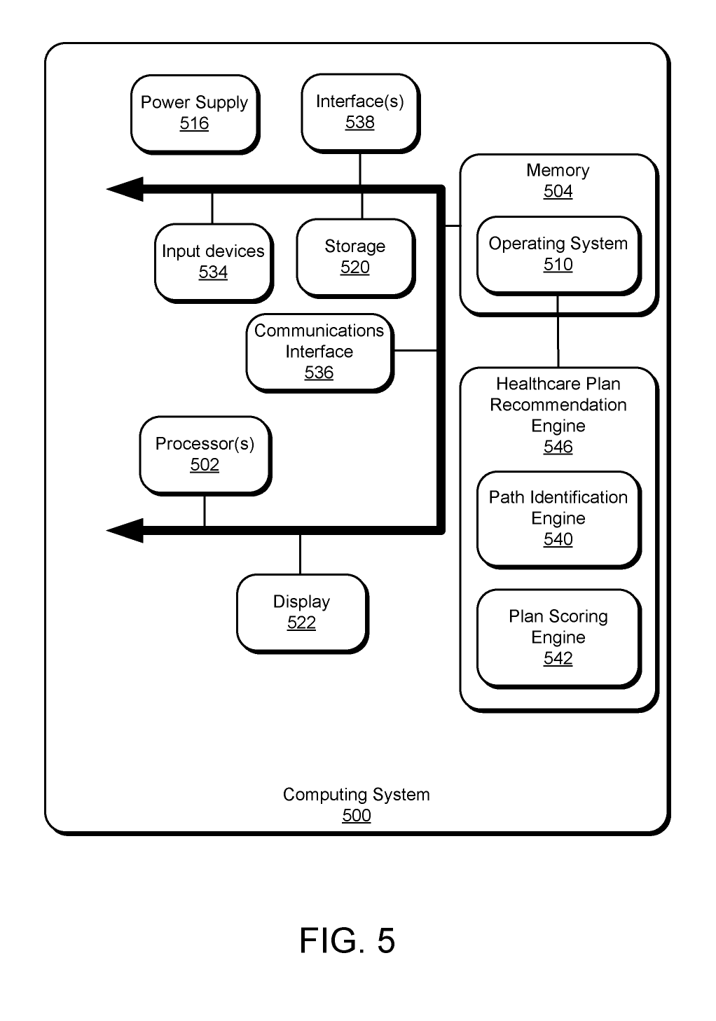

FIG. 5 illustrates an example computing system 500 suitable for implementing one or more embodiments of the disclosed technology. The computing system 500 may be a client device, such as a laptop, mobile device, desktop, tablet, or a server/cloud device. The computing system 500 includes one or more processor(s) 502, and a memory 504. The memory 504 generally includes both volatile memory (e.g., RAM) and non-volatile memory (e.g., flash memory). An operating system 510 resides in the memory 504 and is executed by the processor(s) 502.

One or more modules or segments, such as a healthcare plan recommendation engine 546 are loaded into the operating system 510 on the memory 504 and/or storage 520 and executed by the processor(s) 502. The modules may include the healthcare plan recommendation engine 546 implemented by a path identification engine 540 and a plan scoring engine 542. The path identification engine 540 identifies one or more nodes in one or more hierarchical layered graphs storing historical healthcare claims data based on contextual data associated with employees. The path identification engine 540 also identifies one or more path between the identified nodes based on employer intents or employee intents. The plan scoring engine 542 scores healthcare plans using the one or more hierarchical layered graphs by applying contextual data associated with the plurality of healthcare plans to the identified paths.

Data such as user preferences, hardware configurations, and hardware responses may be stored in the memory 504 or storage 520 and may be retrievable by the processor(s) 502 for use by the healthcare plan recommendation engine 546, the path identification engine 540, and the plan scoring engine 542. The storage 520 may be local to the computing system 500 or may be remote and communicatively connected to the computing system 500 and may include another server. The storage 520 may store resources that are requestable by client devices (not shown).

The computing system 500 includes a power supply 516, which is powered by one or more batteries or other power sources and which provides power to other components of the computing system 500. The power supply 516 may also be connected to an external power source that overrides or recharges the built-in batteries or other power sources.

The computing system 500 may include one or more communication transceivers which may be connected to one or more antenna(s) to provide network connectivity (e.g., mobile phone network, Wi-Fi®, Bluetooth®) to one or more other servers and/or client devices (e.g., mobile devices, desktop computers, or laptop computers) through a communications interface 536. The computing system 500 may further include a network adapter, which is a type of communication device. The computing system 500 may use the adapter and any other types of communication devices for establishing connections over a wide-area network (WAN) or local-area network (LAN). It should be appreciated that the network connections shown are exemplary and that other communications devices and means for establishing a communications link between the computing system 500 and other devices may be used.

The computing system 500 may include one or more input devices 534 such that a user may enter commands and information (e.g., a keyboard or mouse). These and other input devices may be coupled to the server by one or more interfaces 538 such as a serial port interface, parallel port, or universal serial bus (USB). The computing system 500 may further include a display 522 such as a touch screen display.

The computing system 500 may include a variety of tangible processor-readable storage media and intangible processor-readable communication signals. Tangible processor-readable storage can be embodied by any available media that can be accessed by the computing system 500 and includes both volatile and nonvolatile storage media, removable and non-removable storage media. Tangible processor-readable storage media excludes intangible communications signals and includes volatile and nonvolatile, removable and non-removable storage media implemented in any method or technology for storage of information such as processor-readable instructions, data structures, program modules or other data. Tangible processor-readable storage media includes, but is not limited to, RAM, ROM, EEPROM, flash memory or other memory technology, CDROM, digital versatile disks (DVD) or other optical disk storage, magnetic cassettes, magnetic tape, magnetic disk storage or other magnetic storage devices, or any other tangible medium which can be used to store the desired information and which can be accessed by the computing system 500. In contrast to tangible processor-readable storage media, intangible processor-readable communication signals may embody processor-readable instructions, data structures, program modules or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include signals traveling through wired media such as a wired network or direct-wired connection, and wireless media such as acoustic, RF, infrared, and other wireless media.

The implementations of the invention described herein are implemented as logical steps in one or more computer systems. The logical operations of the present invention are implemented (1) as a sequence of processor-implemented steps executed in one or more computer systems and (2) as interconnected machine or circuit modules within one or more computer systems. The implementation is a matter of choice, dependent on the performance requirements of the computer system implementing the invention. Accordingly, the logical operations making up the implementations of the invention described herein are referred to variously as operations, steps, objects, or modules. Furthermore, it should be understood that logical operations may be performed in any order, adding and omitting as desired, unless explicitly claimed otherwise or a specific order is inherently necessitated by the claim language.

Data storage and/or memory may be embodied by various types of storage, such as hard disk media, a storage array containing multiple storage devices, optical media, solid-state drive technology, ROM, RAM, and other technology. The operations may be implemented in firmware, software, hard-wired circuitry, gate array technology and other technologies, whether executed or assisted by a microprocessor, a microprocessor core, a microcontroller, special purpose circuitry, or other processing technologies. It should be understood that a write controller, a storage controller, data write circuitry, data read and recovery circuitry, a sorting module, and other functional modules of a data storage system may include or work in concert with a processor for processing processor-readable instructions for performing a system-implemented process.

For purposes of this description and meaning of the claims, the term “memory” (e.g., memory 504) means a tangible data storage device, including non-volatile memories (such as flash memory and the like) and volatile memories (such as dynamic random-access memory and the like). The computer instructions either permanently or temporarily reside in the memory, along with other information such as data, virtual mappings, operating systems, applications, and the like that are accessed by a computer processor to perform the desired functionality. The term “memory” or “storage medium” expressly does not include a transitory medium such as a carrier signal, but the computer instructions can be transferred to the memory wirelessly.

An example method for generating quantitative recommendation for healthcare benefits plans using hierarchical layered graphs is provided. The method includes identifying one or more nodes in one or more hierarchical layered graphs storing historical healthcare claims data. The one or more nodes are identified based on contextual data associated with one or more employees and on similarities between node contextual data associated with the one or more nodes and the contextual data associated with the one or more employees. The method also includes identifying one or more paths among the one or more nodes of the one or more hierarchical layered graphs based on employer intents of an employer or employee intents of the one or more employees. The method further includes scoring a plurality of healthcare plans using the one or more hierarchical layered graphs by applying contextual data associated with the plurality of healthcare plans to the identified paths. A subset of the plurality of the healthcare plans are identified based on the scoring of the plurality of healthcare plans and a likelihood of selection of one or more of the subset of the plurality of healthcare plans by at least one of the employees. The subset of the plurality of healthcare plans are recommended to the employer and at least one of the subset of the plurality of healthcare plans to one or more of the employees, the at least one of the subset based on the employee intents of the one or more employees.

A method of any previous method is provided, where the method further includes updating the one or more hierarchical layered graphs based on healthcare claims data of the employees.

A method of any previous method is provided, where the method further includes building the one or more hierarchical layered graphs based on the historical healthcare claims data.

A method of any previous method is provided, where the method further includes identifying the employer intents or the employee intents using intent hierarchical layered graphs.

A method of any previous method is provided, where the one or more paths between the one or more nodes are further identified based on the most likely healthcare claims by the one or more employees.

A method of any previous method is provided, where the plurality of healthcare plans are scored further based on the employer intents.

A method of any previous method is provided, where the one or more nodes in the one or more hierarchical layered graphs are identified based further on contextual data associated with the employer.

An example system includes means for identifying one or more nodes in one or more hierarchical layered graphs storing historical healthcare claims data. The one or more nodes identified based on contextual data associated with one or more employees and on similarities between node contextual data associated with the one or more nodes and the contextual data associated with the one or more employees. The system also includes means for identifying one or more paths among the one or more nodes of the one or more hierarchical layered graphs based on employer intents of an employer or employee intents of the one or more employees. The system further includes means for scoring a plurality of healthcare plans using the one or more hierarchical layered graphs by applying contextual data associated with the plurality of healthcare plans to the identified paths. The system also includes means for identifying a subset of the plurality of the healthcare plans based on the scoring of the plurality of healthcare plans and a likelihood of selection of one or more of the subset of the plurality of healthcare plans by at least one of the employees. The system also includes means for recommending the subset of the plurality of healthcare plans to the employer and at least one of the subset of the plurality of healthcare plans to one or more of the employees, the at least one of the subset based on the employee intents of the one or more employees.

An example system of any preceding system further includes means for updating the one or more hierarchical layered graphs based on healthcare claims data of the employees.

An example system of any preceding system further includes means for building the one or more hierarchical layered graphs based on the historical healthcare claims data.

An example system of any preceding system further includes means for identifying the employer intents or the employee intents using intent hierarchical layered graphs.

An example system of any preceding system is provided, where the one or more paths between the one or more nodes are further identified based on the most likely healthcare claims by the one or more employees.

An example system of any preceding system is provided, where the plurality of healthcare plans are scored further based on the employer intents.

An example system of any preceding system is provided, where the one or more nodes in the one or more hierarchical layered graphs are identified based further on contextual data associated with the employer.

An example system for generating quantitative recommendations for healthcare benefits plans using hierarchical layered graphs is provided. The system includes a path identification engine configured to identify one or more nodes in one or more hierarchical layered graphs storing historical healthcare claims data. The one or more nodes are identified based on contextual data associated with one or more employees and on similarities between information associated with the one or more nodes and the contextual data associated with the one or more employees. The path identification engine is further configured to identify one or more paths among the one or more nodes of the one or more hierarchical layered graphs based on employer intents of an employer or employee intents of the one or more employees. The system also includes a plan scoring engine configured to score a plurality of healthcare plans using the one or more hierarchical layered graphs by applying contextual data associated with the plurality of healthcare plans to the identified paths. The plan scoring engine is also configured to identify a subset of the plurality of the healthcare plans based on the scoring of the plurality of healthcare plans and a likelihood of selection of one or more of the subset of the plurality of healthcare plans by at least one of the employees. The plan scoring is engine further configured to recommend the subset of the plurality of healthcare plans to the employer and at least one of the subset of the plurality of healthcare plans to one or more employees, the at least one of the subset based on the employee intents of the one or more employees.

An example system of any preceding system further includes a feedback monitoring manager configured to update the one or more hierarchical layered graphs based on healthcare claims data of the employees.

An example system of any preceding system is provided, where the path identification engine is further configured to build the one or more hierarchical layered graphs based on the historical healthcare claims data.

An example system of any preceding system further includes an intent module configured to identify the employer intents or the employee intents using intent hierarchical layered graphs.

An example system of any preceding system is provided, where the path identification engine is further configured to identify the one or more nodes based on the most likely healthcare claims by the one or more employees.

An example system of any preceding system is provided, where the plan scoring engine is further configured to score the plurality of healthcare plans further based on the employer intents.

An example system of any preceding system is provided, where the path identification engine is further configured to identify the one or more nodes in the one or more hierarchical layered graphs based further on contextual data associated with the employer.

Example one or more tangible processor-readable storage media are embodied with instructions for executing on one or more processors and circuits of a computing device a process including identifying one or more nodes in one or more hierarchical layered graphs storing historical healthcare claims data. The one or more nodes are identified based on contextual data associated with one or more employees and on similarities between node contextual data associated with the one or more nodes and the contextual data associated with the one or more employees. The process also includes identifying one or more paths among the one or more nodes of the one or more hierarchical layered graphs based on employer intents of an employer or employee intents of the one or more employees. The process further includes scoring a plurality of healthcare plans using the one or more hierarchical layered graphs by applying contextual data associated with the plurality of healthcare plans to the identified paths. A subset of the plurality of the healthcare plans are identified based on the scoring of the plurality of healthcare plans and a likelihood of selection of one or more of the subset of the plurality of healthcare plans by at least one of the employees. The subset of the plurality of healthcare plans are recommended to the employer and at least one of the subset of the plurality of healthcare plans to one or more of the employees, the at least one of the subset based on the employee intents of the one or more employees.

Another example one or more tangible processor-readable storage media are embodied with instructions for executing on one or more processors and circuits of a device a process of any preceding process, further including updating the one or more hierarchical layered graphs based on healthcare claims data of the employees.

Another example one or more tangible processor-readable storage media are embodied with instructions for executing on one or more processors and circuits of a device a process of any preceding process, further including building the one or more hierarchical layered graphs based on the historical healthcare claims data.

Another example one or more tangible processor-readable storage media are embodied with instructions for executing on one or more processors and circuits of a device a process of any preceding process, further including identifying the employer intents or the employee intents using intent hierarchical layered graphs.

Another example one or more tangible processor-readable storage media are embodied with instructions for executing on one or more processors and circuits of a device a process of any preceding process where the plurality of healthcare plans are scored further based on the employer intents.

Another example one or more tangible processor-readable storage media are embodied with instructions for executing on one or more processors and circuits of a device a process of any preceding process where the one or more nodes in the one or more hierarchical layered graphs are identified based further on contextual data associated with the employer.

The above specification, examples, and data provide a complete description of the structure and use of exemplary implementations of the invention. Since many implementations of the invention can be made without departing from the spirit and scope of the invention, the invention resides in the claims hereinafter appended. Furthermore, structural features of the different implementations may be combined in yet another implementation without departing from the recited claims. While embodiments and applications of this invention have been shown, and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. A method for generating quantitative recommendations for healthcare benefits plans using hierarchical layered graphs, the method comprising:
identifying one or more nodes in one or more hierarchical layered graphs storing historical healthcare claims data, the one or more nodes identified based on contextual data associated with one or more employees and on similarities between node contextual data associated with the one or more nodes and the contextual data associated with the one or more employees;
identifying one or more paths among the one or more nodes of the one or more hierarchical layered graphs based on employer intents of an employer or employee intents of the one or more employees;
scoring a plurality of healthcare plans using the one or more hierarchical layered graphs by applying contextual data associated with the plurality of healthcare plans to the identified paths;
identifying a subset of the plurality of the healthcare plans based on the scoring of the plurality of healthcare plans and a likelihood of selection of one or more of the subset of the plurality of healthcare plans by at least one of the employees; and
recommending the subset of the plurality of healthcare plans to the employer and at least one of the subset of the plurality of healthcare plans to one or more of the employees, the at least one of the subset based on the employee intents of the one or more employees.

2. The method of claim 1, further comprising:
updating the one or more hierarchical layered graphs based on healthcare claims data of the employees.

3. The method of claim 1, further comprising:
building the one or more hierarchical layered graphs based on the historical healthcare claims data.

4. The method of claim 1, further comprising:
identifying the employer intents or the employee intents using intent hierarchical layered graphs.

5. The method of claim 1, wherein the one or more paths between the one or more nodes are further identified based on the most likely healthcare claims by the one or more employees.

6. The method of claim 1, wherein the plurality of healthcare plans are scored further based on the employer intents.

7. The method of claim 1, wherein the one or more nodes in the one or more hierarchical layered graphs are identified based further on contextual data associated with the employer.

8. A system for generating quantitative recommendations for healthcare plans using hierarchical layered graphs, the system comprising:
a path identification engine configured to identify one or more nodes in one or more hierarchical layered graphs storing historical healthcare claims data, the one or more nodes identified based on contextual data associated with one or more employees and on similarities between information associated with the one or more nodes and the contextual data associated with the one or more employees, the path identification engine further configured to identify one or more paths among the one or more nodes of the one or more hierarchical layered graphs based on employer intents of an employer or employee intents of the one or more employees; and
a plan scoring engine configured to score a plurality of healthcare plans using the one or more hierarchical layered graphs by applying contextual data associated with the plurality of healthcare plans to the identified paths and to identify a subset of the plurality of the healthcare plans based on the scoring of the plurality of healthcare plans and a likelihood of selection of one or more of the subset of the plurality of healthcare plans by at least one of the employees, the plan scoring engine further configured to recommend the subset of the plurality of healthcare plans to the employer and at least one of the subset of the plurality of healthcare plans to one or more employees, the at least one of the subset based on the employee intents of the one or more employees.

9. The system of claim 8, further comprising:
a feedback monitoring manager configured to update the one or more hierarchical layered graphs based on healthcare claims data of the employees.

10. The system of claim 8, wherein the path identification engine is further configured to build the one or more hierarchical layered graphs based on the historical healthcare claims data.

11. The system of claim 8, further comprising:
an intent module configured to identify the employer intents or the employee intents using intent hierarchical layered graphs.

12. The system of claim 8, wherein the path identification engine is further configured to identify the one or more nodes based on the most likely healthcare claims by the one or more employees.

13. The system of claim 8, wherein the plan scoring engine is further configured to score the plurality of healthcare plans further based on the employer intents.

14. The system of claim 8, wherein the path identification engine is further configured to identify the one or more nodes in the one or more hierarchical layered graphs based further on contextual data associated with the employer.

15. One or more tangible processor-readable storage media embodied with instructions for executing on one or more processors and circuits of a computing device a process comprising:
identifying one or more nodes in one or more hierarchical layered graphs storing historical healthcare claims data, the one or more nodes identified based on contextual data associated with one or more employees and on similarities between node contextual data associated with the one or more nodes and the contextual data associated with the one or more employees;
identifying one or more paths among the one or more nodes of the one or more hierarchical layered graphs based on employer intents of an employer or employee intents of the one or more employees;
scoring a plurality of healthcare plans using the one or more hierarchical layered graphs by applying contextual data associated with the plurality of healthcare plans to the identified paths;
identifying a subset of the plurality of the healthcare plans based on the scoring of the plurality of healthcare plans and a likelihood of selection of one or more of the subset of the plurality of healthcare plans by at least one of the employees; and
recommending the subset of the plurality of healthcare plans to the employer and at least one of the subset of the plurality of healthcare plans to one or more of the employees, the at least one of the subset based on the employee intents of the one or more employees.

16. The one or more tangible processor-readable storage media of claim 15, the process further comprising:
updating the one or more hierarchical layered graphs based on healthcare claims data of the employees.

17. The one or more tangible processor-readable storage media of claim 15, the process further comprising:
building the one or more hierarchical layered graphs based on the historical healthcare claims data.

18. The one or more tangible processor-readable storage media of claim 15, the process further comprising:
identifying the employer intents or the employee intents using intent hierarchical layered graphs.

19. The one or more tangible processor-readable storage media of claim 15, wherein the plurality of healthcare plans are scored further based on the employer intents.

20. The one or more tangible processor-readable storage media of claim 15, wherein the one or more nodes in the one or more hierarchical layered graphs are identified based further on contextual data associated with the employer.
